(19) United States
(12) Patent Application Publication
(10) Pub. No.: US 2024/0164674 A1
(43) Pub. Date: May 23, 2024

(54) System for Detecting Mental and/or Physical State of Human

(71) Applicant: Discern Science International, Inc., Tucson, AZ (US)

(72) Inventors: Sailesh Saxena, Bellaire, TX (US); Aaron Elkins, San Diego, CA (US)

(73) Assignee: Discern Science International, Inc., Tucson, AZ (US)

(21) Appl. No.: 18/512,429

(22) Filed: Nov. 17, 2023

Related U.S. Application Data
(60) Provisional application No. 63/384,190, filed on Nov. 17, 2022.

(51) Int. Cl. A61B 5/16; A61B 5/00; A61B 5/0205; A61B 5/024; A61B 5/11; G16H 10/20; G16H 10/60; G16H 50/20; G16H 80/00

(52) U.S. Cl. CPC A61B 5/165; A61B 5/0205; A61B 5/6898; G16H 10/20; G16H 10/60; G16H 50/20; G16H 80/00; A61B 5/024; A61B 5/11; A61B 5/163; A61B 2560/0443; A61B 2560/0462

(57) ABSTRACT

A distributed system for conducting an automatic behavioral analysis for the assessment of the mental state of a human interviewee that includes a human interface system including sensors for generating interview data associated the interviewee; a state assessment server system including a database storing data generated during a plurality of interviews of a given interviewee at different times; and a communication link permitting bi-directional communications between the human interface system and the state assessment server system; where the at least one state assessment server is configured to identify at least one pattern within the stored data to predict the onset of manic/depressive episode for the interviewee.

### CROSS REFERENCE TO RELATED APPLICATIONS

Not applicable

### STATEMENT REGARDING FEDERALLY SPONSORED RESEARCH OR DEVELOPMENT

Not applicable.

### REFERENCE TO APPENDIX

Not applicable.

### BACKGROUND OF THE DISCLOSURE

Field of the Invention: The present disclosure relates to systems for assessing the underlying state of a human. In one embodiment, the present disclosure relates to a system for assessing the underlying mental or physical state of an individual.

Description of the Related Art: Accurate knowledge of the underlying state of a human being can be important for a variety of reasons.

For example, being able to detect the state of a human being suffering from a mental disorder, such as bipolar disorder can be important in identifying, treating, and maintaining suitable conditions for an individual suffering from bipolar disorder (BD). BD is a devastating, lifelong illness characterized by frequent mood episodes, increased suicide risk, increased morbidity, and functional impairment.

A significant challenge with assessing the state of an individual suffering from BD is that accurate diagnosis of BD in youth is challenging since clinical presentations of mania and depression may be difficult to ascertain across different age groups. A further challenge is that it is often difficult to detect the onset of BD because signs of mania and/or depression can occur—in some instances—over time such that a temporally isolated assessment of a given individual may not be able to provide the information required to confirm the diagnosis, whereas the consideration of multiple assessments over an extended period may permit a proper diagnosis. The same is true with other mental illness conditions.

As another example, different mental conditions and states can be evidenced by a significant change in an individual's condition over a baseline, where the individual's baseline condition is subject to gradual change. For such conditions and states regular state assessment of the individual is required to generate an accurate baseline, but such regular assessment is impractical given conventional assessment approaches which typically require direct interaction between the subject individual and a trained and skilled clinician.

Despite the importance of being able to accurately assess the underlying state of human beings, prior attempts to do so have been limited both in terms of their likelihood of success and/or the challenges required for their implementation.

For example, human determination of the underlying state of another human being is generally inaccurate across many situations. While it is true that some individual humans have heightened skills with respect to accessing the state of others, their capacity is not typically applicable to a broad range of individuals from diverse cultural backgrounds or experiences. Thus, a clinician capable of detecting deception with respect to individuals of a certain age and with a certain background history, may not be as successful in assessing the underlying state of an individual of a different age range, with a diverse cultural background.

Another issue with human-based human state assessment is that it is not easily scalable. Even of one were to locate individual humans with a heightened capacity to assess the underlying state of others, it would be difficult, expensive, and practically impossible to deploy them in all situations. A still further deficiency of human-based assessment is that human assessors often have “blind-spots” when it comes to certain individuals or individual types.

To overcome some of the limitations imposed by human-based systems, approaches have been developed to utilize various technologies to assist in the determination of the underlying state of a human being. However, such attempts have been hampered by many of the same challenges posed by human-based approaches.

It is an object of the disclosed subject matter to overcome the described and other limitations of the prior art.

### BRIEF SUMMARY OF THE INVENTION

A brief non-limiting summary of one of the many possible embodiments of the inventions disclosed herein is a distributed system for conducting an automatic behavioral analysis for the assessment of the mental state of a human interviewee, the system comprising: at least one human interface system, the at least one human interface system comprising sensors for generating interview data associated with exhibited attributes of the interviewee; at least one state assessment server system, the at least one state assessment server comprising a database storing data generated during a plurality of interviews of a given interviewee at different times; and a communication link permitting bi-directional communications between the at least one human interface system and the at least one state assessment server system; wherein the at least one state assessment server is configured to identify at least one pattern within the stored data to predict the onset of manic/depressive episode for the interviewee.

None of these brief summaries of the inventions is intended to limit or otherwise affect the scope of what has been disclosed and enabled or the appended claims, and nothing stated in this Brief Summary of the Invention is intended as a definition of a claim term or phrase or as a disavowal or disclaimer of claim scope.

### BRIEF DESCRIPTION OF THE DRAWINGS

The following figures form part of the disclosure of inventions and are included to demonstrate further certain aspects of the inventions. The inventions may be better understood by reference to one or more of these figures in combination with the detailed description of certain embodiments presented herein.

FIG. 1 illustrates one exemplary embodiment of a distributed system constructed in accordance with certain teachings of this disclosure that may be used to conduct an automatic behavioral analysis for personalizing assessment, monitoring, and/or treatment of the mental or physical state of an individual believed to be suffering from a mental or physical disorder, such as bipolar disorder, suicidal ideation or other condition that affects the cognitive processing or state of an individual (e.g., Alzheimer's Disease).

FIG. 2 illustrates an exemplary embodiment of a human interface system taking the form of a generally mobile integrated local appliance.

FIG. 3 illustrates the high-level general operating process of an exemplary distributed state assessment system with respect to a given human interface unit.

FIG. 4 illustrates an exemplary interview process using a touchscreen start button to start the interview and where the virtual interview agent presented to the interviewee is revered to as AVATAR.

FIG. 5, illustrates an embodiment of aspects of the present disclosure in which a child/adolescent will be seated in front of, and interact with, a device which looks like a tablet.

While the inventions disclosed herein are susceptible to various modifications and alternative forms, only a few specific embodiments have been shown by way of example in the drawings and are described in more detail below. The figures and detailed descriptions of these embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts illustrated and taught by the specific embodiments.

### DETAILED DESCRIPTION

The Figures described above, and the written description of specific structures and functions below, are not presented to limit the scope of the inventions disclosed or the scope of the appended claims. Rather, the Figures and written description are provided to teach a person skilled in this art to make and use the inventions for which patent protection is sought.

A person of skill in this art that has benefit of this disclosure will understand that the inventions are disclosed and taught herein by reference to specific embodiments, and that these specific embodiments are susceptible to numerous and various modifications and alternative forms without departing from the inventions we possess. For example, and not limitation, a person of skill in this art that has benefit of this disclosure will understand that Figures and/or embodiments that use one or more common structures or elements, such as a structure or an element identified by a common reference number, are linked together for all purposes of supporting and enabling our inventions, and that such individual Figures or embodiments are not disparate disclosures. A person of skill in this art that has benefit of this disclosure immediately will recognize and understand the various other embodiments of our inventions having one or more of the structures or elements illustrated and/or described in the various linked embodiments. In other words, not all possible embodiments of our inventions are described or illustrated in this application, and one or more of the claims to our inventions may not be directed to a specific, disclosed example. Nonetheless, a person of skill in this art that has benefit of this disclosure will understand that the claims are fully supported by the entirety of this disclosure.

Those people skilled in this art will appreciate that not all features of a commercial embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer's ultimate goal for the commercial embodiment. Such implementation-specific decisions may include, and likely are not limited to, compliance with system-related, business-related, government-related, and other constraints, which may vary by specific implementation, location and from time to time. While a developer's efforts might be complex and time-consuming in an absolute sense, such efforts would be, nevertheless, a routine undertaking for those of skill in this art that have benefit of this disclosure.

Further, the use of a singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the scope of what is claimed.

Reference throughout this disclosure to “one embodiment,” “an embodiment,” or similar language means that a particular feature, structure, or characteristic described in connection with the embodiment is included in at least one of the many possible embodiments of the present inventions. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

The description of elements in each Figure may refer to elements of proceeding Figures. Like numbers refer to like elements in all figures, including alternate embodiments of like elements. In some possible embodiments, the functions/actions/structures noted in the figures may occur out of the order noted in the block diagrams and/or operational illustrations. For example, two operations shown as occurring in succession, in fact, may be executed substantially concurrently or the operations may be executed in the reverse order, depending upon the functionality/acts/structure involved.

Turning now to several descriptions, with reference to figures, or particular embodiments incorporating one or more aspects of the disclosed inventions

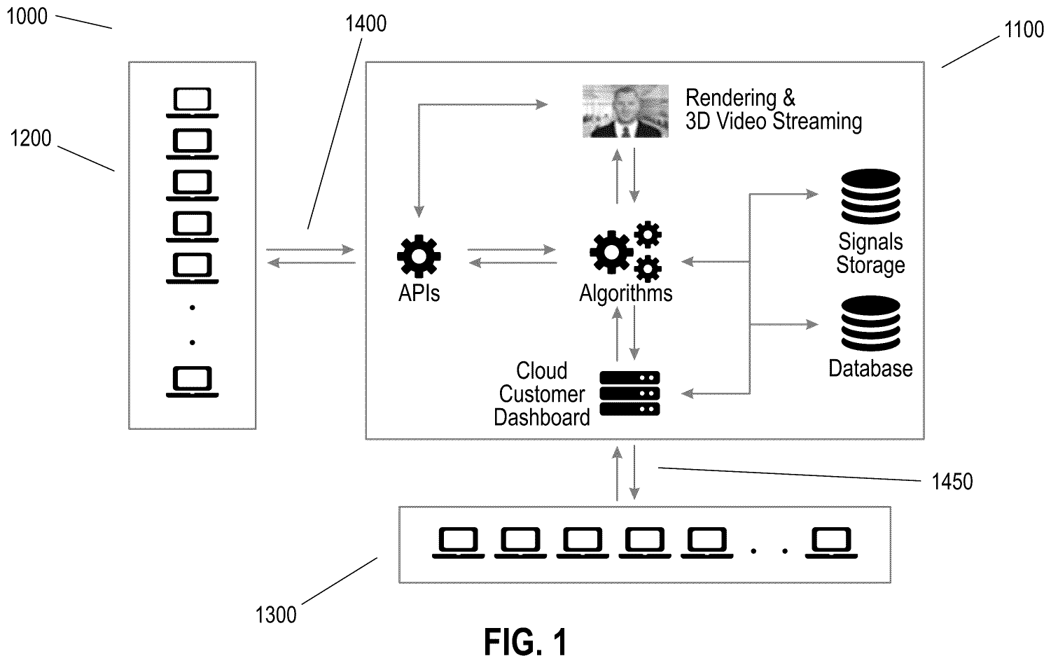

FIG. 1 one exemplary embodiment of a distributed system 1000 constructed in accordance with certain teachings of this disclosure that may be used to conduct an automatic behavioral analysis for personalizing assessment, monitoring, and/or treatment of the mental or physical state of an individual believed to be suffering from a mental or physical disorder, such as bipolar disorder, suicidal ideation or other condition that affects the cognitive processing or state of an individual (e.g., Alzheimer's Disease).

As illustrated in the figure, in this general exemplary embodiment, the distributed state assessment system comprises three main components: (a) one or more state assessment server systems (identified by the box labeled 1100); (b) a plurality of human interface systems (identified by the computer-like images in the box labeled 1200); and (c) one or more communication networks 1400, 1450 permitting bi-directional communication between the state assessment server system (or systems) and the human interface systems (identified by the gray bi-directional arrows between the human interface systems and the state assessment server system(s). In the exemplary embodiment, apparatuses 1300 are also provided to allow authorized persons and entities to access the state assessment server system for modifying or adjusting the state assessment server system, receiving reports concerning the operation of the system, receiving analysis and/or reports concerning one or multiple human interactions conducted by the system, or for any other purpose.

In the exemplary embodiment, each of the human interface systems 1200 is used to initiate an interaction with a specific human. For purposes of this disclosure, a discrete grouping of interactions between the distributed state assessment system and a human individual is referred to as an “interview” and the human individual involved in a given interview is referred to as an “interviewee.”

An interview may consist of one or a more different interactions between the state assessment system and a given interviewee, and that the interactions may take the form of the provision of various different stimuli to the interviewee and the detection of the response (or responses to those stimuli). For example, in one form, an interview could involve interactions where questions are visually presented to an interviewee and the interviewee is requested to respond by typing answers on a keyboard. In other embodiments, the interview could take the form of an interactive interface that utilizes a virtual person to conduct a verbal question-and-answer interrogation of the interviewee, where questions are posed verbally, and the interviewee is requested to articulate their response. In still other examples, the interactions could take the form of the presentations of images, sounds, smells, or the like to the interviewee and the determination of the interviewee's responses to those stimuli. Still further, the interactions comprising an interview could take the form of a combination of the above-described and other forms of stimuli.

In addition to presenting the communications giving rise to the interview to the interviewee, the human interface system 1000 will also detect certain reactions of the interviewee to the stimuli. For example, the human interface system can include microphones to capture the interviewee's audible response to presented questions. It can also include a camera and an eye tracker for detecting the interviewee's posture and eye gaze during the interview. It could also include a variety of other detectors and sensors for detecting other responses from the interviewee, such as posture changes, pulse rate changes, changes in skin activity (e.g., pore opening, sweating, temperature changes, etc.). As described in more detail below, the human interface system(s) will transmit data reflecting the sensed and detected attributes of the interviewee to one or more state assessment server systems.

In the illustrated embodiment, the state assessment server system(s) 1100 interacts with the human interface systems (through the communication system(s)) in such a manner that the state assessment server system 1100 determines a variety of desired interactions for a given interview. These interactions can either be scripted—in the sense that for a certain interviewees or groups of interviewees—the same series of questions can always be presented in the same order or dynamic. In a dynamic interview, for a given interviewee, the state assessment server system(s) will request the human interface system to establish certain interactions with the interviewee and will then use the responses received from certain initial interactions to determine which (if any) subsequent interactions to request.

At a high level, the operation of this exemplary illustrated embodiment is as follows. At a first time a human interviewee will interact with a specific one of the human interface systems 1200 in a manner that causes the involved human interface system to send a notice to one or more of the state assessment server systems 1100 that it is time to initiate an interview. In response to the signal, one or more of the state assessment server system(s) will cause to be communicated to the specific local device requests for one or more interactions and (in some embodiments) requests for certain detected data.

The requested interactions (e.g., a requested series of questions and video displays) will then be presented to the interviewee by the specific human interface system 1200 and the interviewee will interact with the human interface system in response to the one or more initial interactions. The response (or responses) received by the human interface system will then be transmitted from the human interface system to the state assessment server system 1100 (either with or without some local processing). The state assessment server system 1100 will then receive and process the received response(s) and, in response, may generate a subsequent set of requested interactions to be transmitted to the human interface system. The human interface system 1100 can then present the subsequent interactions to the interviewee and receive responses from the interviewee. The process may be repeated several times with a number of subsequent interactions presented to the interviewee and a number of subsequent responses received by the state assessment server system.

In the above example, the state assessment server system 1100 can then process the received response to provide a general assessment of the underlying state of the interviewee.

In one embodiment the interview is arranged to provide structured questions that can automatically detect subtle physiological signals (beyond the level of human perception) emanating from different mood states such as mania, bipolar disorder (“BD”), depression, suicidal ideation, or states associated with the use of one or more illicit substances (or the failure of a given individual to take certain prescribed medications). The objective is to create diagnostic and therapeutic tools that provide consistent and reliable results using the automated state detection technology.

The interviews conducted by the exemplary system are non-invasive and at a conversational distance. The behavioral sensory cues utilize by the exemplary state assessment system together with clinical assessments will be used to assess mood and behavioral changes in youth with a state condition(s), such as BD, which can lead to diagnostic accuracy and personalized treatment decisions. Results from use of the disclosed exemplary state assessment system can produce well-defined behavioral metrics of affective processes, for example, in youth with state conditions, such as BD, and validated data collection parameters that can be translated directly to research and clinical contexts.

The exemplary state assessment system may take the form of an Al based interviewing system that conducts natural, brief interviews for assessing the mental and/or physical state of an individual at the time of a given interview and/or over the course of multiple interviews.

The exemplary state assessment system 1100 may incorporate an embodied conversational agent (ECA) who conducts a fully automated interview. A patient interacts naturally with the exemplary state assessment system (via voice and in their native language) to complete a self-service clinical interview. The exemplary state assessment system uses multiple non-contact behavioral and physiological sensors to measure the passenger's nonverbal and verbal behavior to assess their mental and behavioral health. The results are then fused, and a report is generated for a human clinician to review and incorporate into their diagnosis and treatment.

The exemplary state assessment system 1100 can provide an ongoing analysis of patient behavioral health by incorporating all previous interviews to conduct multivariate longitudinal probability predictions of negative mental health states. Each patient has their own behavior calibrated against themselves do identify temporal and cross-sectional behavioral health indicators.

In one embodiment, the ECA can take the form of an intelligent virtual human. The use of intelligent virtual human (VH) agents for clinical purposes has led to the creation of highly interactive, artificially intelligent, and natural language capable VHs that can engage real human users in a credible fashion and provide clinical assessments. Virtual humans can perceive and act in a virtual world, engage in face-to-face spoken dialogues, and exhibit believable human-like emotional reactions during interactions with real humans. For example, VH agents can conduct clinically oriented interviews within a safe non-judgmental context which may encourage learning or disclosure of important information.

In certain embodiments the ECA can take the form of an agent tailored to evoke desired interaction with a given interviewee. Thus, for very young children, the ECA can take the form of a cartoon character, an taking animal or another non-human agent with which such a child may easily interact. In other embodiments—for example, an adolescent interviewee—can take the form of a favorite musician, an athlete, or other real person with which the interviewee may be comfortable interacting. In still other embodiments, the ECA can take the form of an idealized person, such as a clinician with a smooth, calming voice. In this way, the interviewee will be placed at ease while they are responding to the scripted questions from the exemplary state assessment system.

Facial expression and body gestures play an important role in human communicative signaling, while vocal characteristics (e.g., prosody, pitch variation, etc.) provide additive information regarding the “state” of the speaker beyond actual language content. The ability of VH agents to analyze non-verbal behavioral signals gleaned from the user's facial expressions, body gestures and vocal parameters can be used to detect intentions, goals, values, and the emotional state of a patient. Inferences from these sensed signals could then be used to supplement information that is garnered exclusively from the literal content of clinical assessments.

One use of the disclosed exemplary state assessment system is for collecting objective and systematic behavioral health information over time during interactions with children and adolescents diagnosed with BD. To accomplish this aim, traditional screenings can be combined with verbal, nonverbal and physiological measures can be used to identify behaviors associated with anxiety, depression, or mania. From these behavioral sensory cues, inferences can be made by the disclosed system to quantify mood and behavioral states across the interactions.

Data acquired from the capture and analysis of verbal, nonverbal and physiological behaviors emitted during interactions with the exemplary state assessment system can be compared/correlated with (1) a structured diagnostic interview, and (2) clinician and self-report measures of mania, depression, anxiety, and attention-deficit hyperactivity disorder. The appearance and demeanor of the exemplary state assessment system can be customized to encourage rapport during the interaction.

As will be appreciated, the disclosed exemplary state assessment system can be used to advance the application of technological devices to objectively recognize the onset and severity of behaviors consistent with a clinical diagnosis of a mental state condition, such as bipolar disorder. This can ultimately advance the use of technologies to identify subpopulations of youths that may differentially respond to a treatment approach. To address this objective, the disclosed state assessment system can implement a consistent exemplary state assessment system protocol to objectively measure emotional processes and behaviors. Such a protocol can be non-invasive, interactive and measure behavioral sensory cues, which can be developed into an asset for clinicians. For example, when a child is being interviewed, behavioral sensory cues can be reliably and automatically detected by the disclosed state access system based on the most subtle physiological responses generated during the interview, which can—in some embodiments—be shared with the parent and/or child.

In one exemplary embodiment, entire interview is conducted through ECA interaction with the interview. In other embodiments, the interview can be conducted by a live, present human interviewer, with the state assessment system used to analyze the responses to the posed questions to aid the interviewer or a clinician assessing the interview. For example, on questioning, the child may respond, “I am OK, I am fine,” to the clinician. However, the exemplary state assessment system may pick up sensory cues that signal the clinician to probe further. This is especially important in situations where suicidal behaviors, substance use problems or other high-risk behaviors are a concern. In this way the exemplary state assessment system can be a valuable partner to the clinician.

In some embodiments, the exemplary state assessment system can be used over time to conduct regular interviews of an interviewee to detect changes that occur over time. At present, in many instances, individuals—primarily youths—often present with an acute onset of manic/depressive symptoms. The precursor to these episodes is not always evident to the child or others around him/her. Use of the disclosed exemplary state assessment system can help create a pattern of consistent sensory cues that can be linked to a pattern of mood symptoms to predict onset of manic/depressive episodes. Importantly, this information may help abort acute manic/depressive episodes through early therapeutic interventions. Thus, disclosed state assessment system can be partnered with clinicians to provide objective, reliable and consistent assessments to improve the diagnosis and treatment of youth with BD such that it aids—but does not supplant—qualified clinicians who work with patients.

In one embodiment, the exemplary state assessment system) which is an embodied conversational agent (ECA) that conducts natural and non-contact interviews. During the interview, the exemplary state assessment system measures Kinesics (motion and gesture); Physiological (heart rate, respiration rate, heat signature); Vocalics (how it is said): Oculometries (eye movement). These behavioral and physiological data, when paired with a consistent interview protocol, facilitate more accurate classification of emotional and cognitive states.

The interview can consist of a carefully created script of questions for the exemplary state assessment system to ask the interviewee (e.g., a child/adolescent or an adult) where each interviewee will be asked to respond to the same scripted questions. These scripted questions can be questions from validated mania and depression rating scales of the type routinely asked during a clinical assessment by our research team. One example of such a script of questions is set out below:

Draft of Scripted Questions:
Q1: Hello, my name is Hanna. What is your name?
T1: I am going to ask you some questions about how you are feeling.
Q2: In the past week how has your mood been?
Q4: In the past week have you had only a little energy or lots of energy?
Q5: Is it hard to sit still and pay attention to what you are doing?
Q6: In the past week, have you been sleeping less than usual?
Q7: Do you want to take a nap during the day?
T2: You did a great job answering my questions. I enjoyed talking with you.

Have a good day.

In one example, the human interface used to conduct the interviews can take the form of a special-purpose table-like appliance having built in sensors that have the capacity to observe and capture objective data signals relating to the interviewee's: Kinesics (motion and gesture); Physiological (heart rate, respiration rate, heat signature); Vocalics (how it is said): Oculometries (eye movement). Such sensors function far beyond human capacity for perception and accuracy. The sensors perform the same from interview to interview and from interviewee to interviewee.

In such an example, the system can then analyze patterns that emerge from individual children/adolescents and develop predictive algorithms that can be improved with use and learning with the intention of creating standardized predictive tools to complement the clinicians in their diagnosis of bipolar disorder.

In some embodiments and applications, a human individual—such as a parent or a psychiatrist—can always be present in the room during the interview with the exemplary state assessment system. In such embodiments, if the interviewee (e.g., the child/adolescent or adult) expresses that they wish to stop the exemplary state assessment system interview, the observer can stop the interview immediately.

During interviews in which a human observer is present (physically or virtually) such observer can affirmatively look for signs and indications that the interviewee (e.g., the child/adolescent or adult) is feeling uncomfortable or is becoming anxious or upset during the interview with the exemplary state assessment system and stop the interview immediately if this occurs. Once the interview is stopped, the individual can ensure the interviewee feels comfortable before the interview is resumed or the interview will not be resumed.

The interactive experience can be tailored to establish an interview environment to mirror a comfortable interaction with an ideal clinician. As will be appreciated, this tailoring can vary from interview type to interview type and from specific interviewee to specific interviewee.

For example, in embodiments where the exemplary state assessment system is to be used with children and/or adolescents, the interview can be designed to be comforting to the interviewee (e.g., the child/adolescent or adult) in every manner. In such embodiments, the exemplary state assessment system will not have an unpleasant appearance or speak aggressively to the interviewee and the goal in such an embodiment will be to never to make the interviewee upset or anxious in any way.

The appearance, demeanor, facial expression, and voice of the exemplary state assessment system can ensure that the personal characteristic of the exemplary state assessment system is designed to be comforting to the interviewee (e.g., the child/adolescent or adult). Therefore, the exemplary state assessment system be designed to assessment system on the DIA will resemble a female with a soft-spoken voice. In this way, the interviewee will be placed at ease while they are responding to the scripted questions from the exemplary state assessment system. Our research team always conducts interviews where the child is always made to feel comfortable.

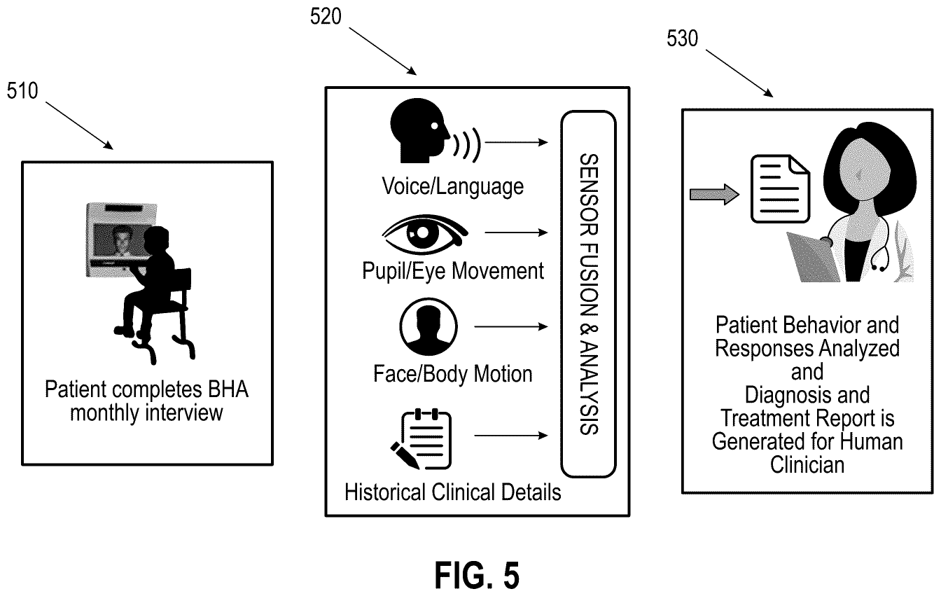

In one embodiment, reflected FIG. 5, a child/adolescent will be seated in front of the device which looks like a table as shown at 510. The single purpose computer/tablet will be turned on. The child/adolescent will be asked to look at the screen and listen to the voice of the exemplary state assessment system. They will be asked to keep looking at the exemplary state assessment system while the exemplary state assessment system is asking them questions. They will be asked to respond the questions as best as they can. The child/adolescent will not touch the screen. This is the only interaction they will have with the exemplary state assessment system.

Inputs will be received by the system and analyzed as reflected at 520 and a report containing an assessment and/or treatment report of the interviewee can be provided. That report can be provided to human clinician for use in interactions with, and potential treatment of, the interviewee as reflected at 530.

By judiciously determining where and how various aspects of the described process are implemented, the embodiments of the present system provide a highly flexible, highly scalable, cost-effective and robust system for discerning the underlying state of humans suitable for a large number of applications.

Various aspects, and several of the many possible alternative embodiments of the exemplary distributed state assessment system will be exemplified below. When considering the following written description, it will be understood by those of skill in the art that the various embodiments are non-limiting and structural components and/or functional characteristics may be combined, a la carte style, to provide systems that have various structural configurations and functionality. For example, and without limitation, as discussed in more detail below, each of the human interface systems in a particular embodiment of the distributed state assessment system may take the form of any of a stationary system, a mobile system, a desktop system, a tablet-based system, or a smartphone system, and other interface systems that may be envisioned by those of ordinary skill in the art. The discussion of an embodiment utilizing desktops is in no way intended to preclude a system that would combine human interface systems that have other forms such as a desktop form, a tablet form, and/or smartphone forms. Those ordinarily skilled in the art may practice the inventions taught and disclosed herein with these and many other forms and combinations. Accordingly, unless explicitly noted otherwise, all exemplary embodiments and all exemplary variant embodiments disclosed herein should be understood to be combinable with all other envisioned embodiments and variants to achieve the stated purposes and results of the inventions described herein.

THE HUMAN INTERFACE SYSTEM: As generally described above, each human interface system of the present disclosure is a system that permits the overall system to interface with one or more human interviewees to both: (a) present stimuli to a human interviewee and (b) receive and detect attributes of a human interviewee, including specifically responses from a human interviewee to provided stimuli.

Stimuli and Output Apparatus: The stimuli provided to each human interviewee, and the apparatus within each human interface system providing such stimuli, can vary depending on the application of the overall system. In a most basic case, the stimuli can consist solely of audible stimuli in the form of questions presented to the human interviewee. In such embodiments, the human interface system may necessarily include one or more audio speakers for providing the audible messages.

In more typical embodiments the stimuli provided by the human interface system may include audible stimuli (described above) and visual stimuli. As with the audible stimuli, the visual stimuli may take various forms including but not limited to words, static images, video clips, holographic images, displayed 2D or 3D images, displayed physical objects, moving apparatuses, a virtual human agent (which could take the form of a 2D or 3D moving image, a hologram, a robot, an animatronic figure, a cartoon-like humanoid character), or any other suitable form.

Detected Attributes and Sensors: The specific interviewee attributes detected by the human interface system will vary depending on the application, the nature of the stimuli provided for a specific embodiment, and other factors, such as cost, size and bandwidth constraints that may be placed on the system. In many preferred embodiments, the detected attributes (and their associated detecting sensors) will be attributes that can be detected non-invasively (i.e., without making physical contact with the human interviewee). Such attributes include, for example, verbal responses, eye movement, general body posture, facial expressions, etc.) In other embodiments, the detected attributes may include (in addition to the non-invasively detectable attribute discussed above) attributes in which some physical contact with the interviewee is required. Such attributes may include, for example, weight/weight-distribution attributes, which require the human interviewee to stand on a force platform or other similar device, and blood-pressure, which may require the interviewee to interact with a pressure cuff. Certain detectable attributes, such as respiration rate, heart rate, and others, can—depending on the nature of the detectors and the processing nature of the system—be detected either non-invasively (i.e., no physical contact with the human interviewee) or invasively.

In a basic case, the human interface system may include detectors for detecting audible/verbal responses from an interviewee, visual information concerning visible aspects of the interviewee and eye movement.

To detect audible/verbal responses a microphone (or microphone array) may be utilized. The received audible data can be analyzed to determine vocalic aspects of the interviewee's responses, such as pitch, pitch-changes, rate of speech, tempo, volume/intensity etc. The received audible data can also be processed to provide linguistic data related to the interviewee's response such as the specific informational content of the verbal response (i.e., what is being said such as “yes,” “uh-hu,” “I don't know,” to much more complicated responses); the extent of pronoun usages as opposed to more specific references such as hedging, avoidance, etc.

To detect visual aspects of the interviewee, one or more cameras may be employed. To detect eye movement, one or more eyer trackers may be utilized. The eye trackers used in the disclosed system may take one of many forms. In certain examples, the eye trackers may be dedicated apparatus built into a specific device. Such dedicated eye trackers may include, for example, eye trackers available from Tobii, Gazepoint, ISCAN or others. In alternate embodiments, the eye trackers may take the form of wearable devices, such as an interactive “glasses” like device or other eyewear.

In more sophisticated and complex embodiments of systems constructed in accordance with the teachings of this disclosure, detectors may be used to detect a large variety of attributes of the human interviewee. A non-exhaustive list of such attributes, along with a brief discussion of exemplary apparatus that may be used to detect such activity are discussed below.

Kinesics Attributes, such body posture, body movement/shifts, limb (e.g., hand or finger) movement, overall posture, etc. Such kinesics attributes can be determined by analyzing video data, receiving force platform data, or both, or a combination.

Eye-Related Attributes (sometimes referred to as ocular-metrics), such as gaze location (i.e., what spot is the human interviewee focusing on); gaze duration (how long is the human interviewee looking at a specific location); pupil dilation; gaze pattern (is the human interviewee scanning visual stimuli in a raster pattern or is their gaze jumping back and forth to and from a single displayed image); and blinking patterns. Such eye-related attributes can be detected using a dedicated pupil sensor, an eye tracking device that can additionally provide information on gaze location and duration, or—in certain embodiments—through processing of high-quality video imaging of the human interviewee.

Temperature: The temperature of the human interviewee at one or various locations can be detected using thermal sensors. These sensors can be either contact sensors (i.e., sensors that contact the human interview) or non-contact sensors.

Dermal, or skin-related, activity, such as skin pore activation, galvanic skin response, variations in sweat-gland activity, skin conductance etc.: The status of the human interviewee's skin pores (for example, the pores on the fact) with respect to whether they are open, closed, or partially open, can be detected through processing of a high-quality video signal focused on an exposed area of the human interviewee's skin (for example the face and more specifically, the cheeks). Other aspects of the human interviewee's skin condition, such as galvanic skin response, can be detected using invasive sensors (e.g., contact electrodes).

Heart rate/pulse: The human interviewee's heart rate can be detected using an invasive sensor (i.e., one requiring contact with the human interviewee) such as a blood pressure cuff or through non-invasive approaches such as analysis of high-quality video or thermal data associated with the interview.

Blood pressure: Blood pressure will typically be measured through the use of an invasive sensor (such as a blood pressure cuff), although aspects of blood pressure can be inferred from analyzing aspects of a high-quality video of the human interviewee during the interview.

Facial movements, including micro-expressions: Facial movements (including micro-expressions) can be detected by analyzing high quality video of the human interviewee.

Body movement, postural anomalies, body rigidity, posture/stance: These body-related attributes can be detected by analyzing a video feed of the human interviewee during the interview, using an apparatus like a force platform that directly detects movement, and/or through a combination of video analysis and direct sensors. In certain alternative embodiments, such body movement attributes may be detected using a wearable device (e.g., a vest-like device) that includes gyroscopic or other sensors capable of providing data pertinent to determining or inferring body movement attributes of an interviewee.

Brain activity: brain activity can be detected directly through an invasive sensor (such as an EEG) or through non-invasive sensors (which can sense for emitted detectable signals or the interaction of a brain-emitted signal with another signal, such as a high-frequency signal emitted by a sensor and detected by a receiver).

Biometrics sensors: In addition to the sensors described above, the human interface system may also include one of more sensors for interacting with the human interviewee in such a way that a unique identity of the interviewee can potentially be verified. Such sensors may include, for example, fingerprint sensors, iris-pattern detectors or other devices for sensing attributes considered to be unique to a given individual.

Document/token sensors: In addition to the sensors described above, the human interface system may also include one of more sensors for reading and/or detecting attributes of documents or tokens associated with a human interviewee. Such sensors may include, for example, document scanners, passport readers, RFID readers, bar-code scanners, magnetic strip readers, etc.

PHYSICAL FORM OF THE HUMAN INTERFACE SYSTEM: The specific form of the human interface system will vary from application to application and need not be consistent within a given implementation of the described overall system. For example, the human interface system may take the form of a generally fixed system that will typically be positioned at one location within a space and will remain at that location for extended periods of time or permanently. The human interface system may also take the form of a mobile system that can be easily moved from location to location as needed for various applications. In yet further examples, the human interface system can take the form of a semi-mobile “home” apparatus that can easily be transported and used in a home location. Further details concerning variant implementations of the human interface system are discussed below:

Generally Fixed Systems: Certain or all the human interface system may take the form of generally fixed systems. Such systems are characterized as “generally fixed” because they are not easily movable. A generally fixed system is not necessarily a system that is affixed to a particular location. As an example, a single large kiosk structure may be described as a “generally fixed” system because it is not easily and quickly movable by a single individual, even though the kiosk may be removable within a given location or space.

Because a generally fixed system will typically be intended for long-term use at a single location for a substantial period of time, such systems may utilize more bulky, expensive, and complex systems that other systems. For example, instead of using a small monitor screen for the display of visual images and videos to a human interview, such a system may use a more complicated display such as a large projection screen, a shaped screen in a humanoid form placed in a three dimensional environment (to provide a more realistic presentation of a virtual interviewer), a hologram, or, in some instances, a animatronic robot, designed to mimic all or part of the body of an actual human. In embodiments where a robot will be used with individuals having special interactive needs (e.g., small children or individuals with various processing difficulties)—such as in a hospital or therapeutic environment—the robot may take the form of an animal-like or cartoon-like character to produce an environment more conducive for that particular type of interview.

Dedicated Distributed Generally Fixed Systems: In certain embodiments, the human interface system may take the form of a dedicated distributed system that includes discrete apparatuses and sensors designed to provide a high-fidelity interview environment and where the responses from the interviewee can be detected with significant precision. Such embodiments may involve the utilization of a dedicated room that includes a number of output apparatus and sensors including some or all of the following: a force platform, multiple speakers arranged for dimensional sound control, multiple high-definition screens to provide visual outputs to the interviewee, actuators to provide kinesthetic or haptic output to the interview (e.g., vibrations, etc.). Since such embodiments may typically involve a specific location and numerous sensors, invasive interface devices such as blood pressure monitors, eye tracking goggles, vests with sensors to detect movement, and the like may be used. By providing such a broad range of output apparatus and detectors, a distributed generally fixed system may provide a virtual reality type experience for the interviewee in which as many variables of the interviewee's sensory experience are controlled and dictated by the user interface system as possible.

Generally Fixed Desktop System: A further version of a generally fixed user interface system may be a desktop-based system. Such a system may include, for example, a dedicated computer that includes a video monitor for providing visual stimuli for use in interviews, such as video of a virtual interview agent. Such a system may also include integrated or separate speakers for audio output and HD camera and a microphone for detecting interviewee responses. In general, such a system may also include an eye tracking device. Generally, fixed desktop systems are of potential benefit for applications where a number of users can easily be directed to the same location for an interview such as, for example, in applications where the device will be used for employment pre-screening and regular post-employment screening of employees.

Mobile Systems: As described herein, a mobile human interface system is a system that is designed to be relatively easy to move when compared to generally fixed systems. Mobile systems can be beneficial in applications where a system may need to be periodically moved, such as in an airport, where security screening stations may need to be moved on an annual (or more frequent) basis to accommodate new equipment or access applications—such as event access at a given local—where the scale of the system may need to be adjusted to address changes in the anticipated number of event attendees. Mobile systems may also be beneficial in applications where the human interface system may need to be regularly moved during operation to either bring the human interface system to a specific user who has disabilities precluding use of a more-fixed system or to a user who is apprehended and stopped in a specific location (e.g., a trespasser caught by a robotically movable human interface system in an unexpected location of a warehouse). As described in more detail below, mobile human interface systems may also be of beneficial use in applications where it is desirable to catch a human interviewee “off-guard” by conducting an interview at a generally unexpected location and time. For example, in an airport security environment, most potential passengers expect to be screened at a dedicated screening checkpoint. Using a mobile human interview, a screening interview can be conducted either prior to the main screening location (e.g., by taking a potential passenger out of the screening line aside for an interview) or long after a potential passenger has cleared the initial screening (e.g., at the gate, just prior to boarding).

Additional details of mobile forms that the human interface system may take are discussed below:

Generally Mobile Integrated Appliance:

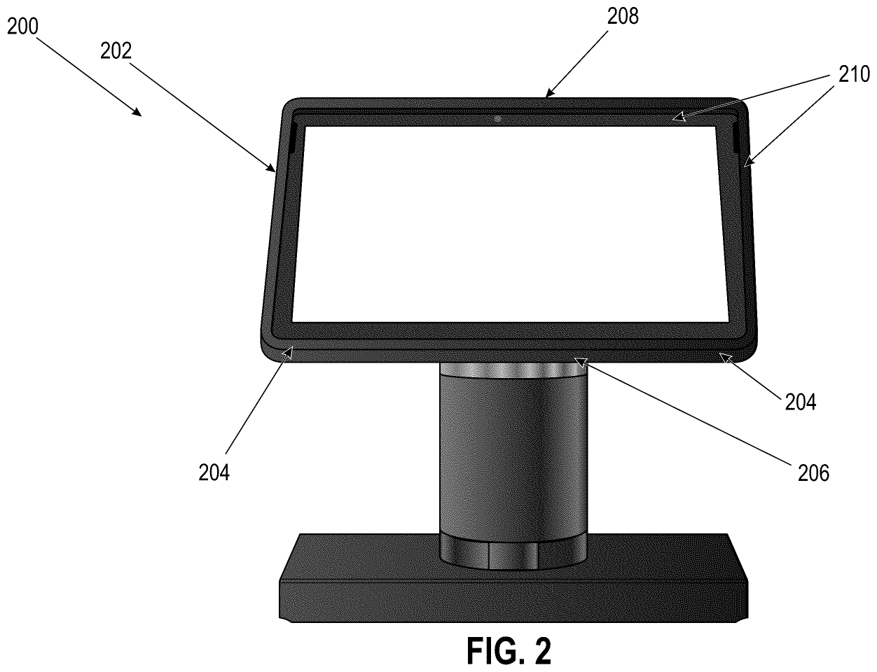

In one embodiment, the human interface system may take the form of a generally mobile integrated appliance where the generally mobile integrated appliance may have a form factor somewhat like a tablet computer. FIG. 2 illustrates an exemplary embodiment of a human interface system taking the form of a generally mobile integrated local appliance 200.

As depicted in FIG. 2, the local appliance 200 may take the form of a robust and rugged tablet computer 202. The local appliance may be of such a size that it is suitable for handheld use and/or for use as a table/desk-mounted or resting device and/or as a device that may be mounted on an articulated arm for variable positioning. When used with a variable arm, the local appliance may either be affixed to the arm or coupled to the arm in such a way that it can easily be de-coupled and moved. In further embodiments, the local appliance may be connected through the arm or an alternate mounting structure to a power supply to eliminate direct dependency on a battery. In many embodiments, the local appliance may be tethered to a non-moving or difficult-to-move structure to reduce the potential that the local appliance will be lost, misplaced, or stolen.

In general, the local appliance will include a processor, which may take the form of a system on a chip (“SOC”) or system on a module (“SOM”) processor and, optionally, one or more application processors, graphical processing units (“GPUs””) and/or hardware accelerators. In embodiments where a SOM device is used, the SOM may take the form of a Qualcomm Snapdragon QCS8250 or similar processor.

In embodiments including a SOM, the SOM may provide the computing, memory, storage, wireless connectivity and the basic I/O functionality for the local appliance. The SOM may also include built-in WiFi/BT circuitry, a MIPI to HDMI bridge, an audio codec & amplifier to interface with a microphone array and speakers & battery management. The SOM may also include I/O ports such as Camera MIPI CSI ports, Display MIPI DSI port, HDMI signals, High Speed PCIe x2, USB3.x, and low speed interfaces like I2C, UARTs & GPIOs that may be terminated on a plug in an edge connector. The SOM may follow the industry standard SMARC form factor. However, the edge connector pin function may be extended to accommodate additional interfaces beyond those defined by the SMARC standard.

The local appliance may also include memory in the form of systems memory and local onboard storage. Embodiments are envisioned wherein the local appliance takes the form of a relatively small, low-cost device, with minimal memory and storage. For example, embodiments are envisioned wherein the system memory is between about 7-10 Gigabytes and the onboard storage is between about 120-140 Gigabytes.

The local appliance may typically also be provided with equipment capable of supporting bi-directional communications with other devices. The specific type of communications enabled by such hardware may vary from implementation to implementation and may, for example, take the form of hardware enabling WLAN, WWAN, WiFi (Dual Band), WiFi 6, Cellular (including 5G), Bluetooth, Ethernet, USB, or similar media.

The local appliance may also include a display, which may take the form of a LED panel with capacitive touch functionality.

The local appliance may also be configured to interact with a number of sensors and detectors to permit the local appliance to detect various attributes and characteristics of an interviewee undergoing an interview. Such sensors and/or detectors may be integrated into the local appliance, or arranged to communicate with the local appliance, using one or more of the communication approaches discussed above. Such sensors and detectors may include, for example, all or a subset of the following components discussed below.

For example, the local appliance may include a High-Definition face streaming camera 208 for detecting the facial movements of the interviewee. While such a camera will typically be integrated into the local appliance, such a camera may take the form of a separate component that provides a data feed to the local appliance via wired or wireless communications. Still further, the local appliance may interact with a plurality of facial streaming cameras, with all or some of the plurality of cameras being integrated into the local appliance. The use of a plurality of face streaming cameras can permit the local appliance to receive data feeds that are either duplicative (for error detection and/or correction) or different. Note that still further embodiments are envisioned where streaming cameras are used to not only detect the facial attributes of the interview but also other attributes of the interviewee during the interview, such as respiration rate (e.g., breaths per minute), overall movements (e.g., still or fidgety), or other visibly observable attributes.

As another example, the local appliance may also include an eye tracker 204. While embodiments are envisioned wherein the same camera used as a face streaming camera provides data for use in connection with eye tracking, for many embodiments, use of a dedicated eye tracker component (which may include one or more cameras and a processor for processing data from such a camera) may be preferred. For example, embodiments are envisioned wherein the eye tracker may be one of the type offered by Tobii that provides real-time data streams corresponding to gaze point, eye position, pupil diameter, user presence, and head pose.

To capture audio inputs, the local appliance may include an integrated microphone 210 and/or be capable of receiving signals form one or more separate external microphones. Such microphones may be general in nature or highly directional. For example, to detect subtle changes in the respiration of the interview, directional microphones targeting the area around the interviewee's nose and mouth may be used. In one embodiment, the local appliance may include a microphone array that can enable accurate audio capture and provide echo and noise cancellation functionality.

To permit audible interaction with the user, the local appliance may also typically include (or be capable of communicating with) an audio speaker 206 or similar device.

A variety of other sensors and/or detectors may be included within the local appliance and/or provided to interact with the local appliance. As one example, the local appliance could include (or be adapted to communicate with) a thermal camera to detect temperature changes with an observed region, such as the front face of an interviewee. The local appliance could also include (or be configured to communicate with) one or more biometric sensors capable of providing biometric information about the interviewee, such as a fingerprint scanner or an iris scanner. Still further one or more physiological sensors could be provided to provide information concerning detectable attributes of the interviewee such as general body temperature, heart rate, pulse, respiration rate, or sweat level. Such added physiological sensors may be external to the local appliance and may interface with the external appliance using wired or wireless technology, such as USB or Bluetooth. Embodiments are envisioned wherein the physiological and other sensors (e.g., a gyroscopic sensor(s) to detect movement) are integrated into a wearable device, such as a vest or jacket, that the interviewee can wear during an interview.

Furthermore, the local appliance may be adapted to communicate with a scale or force platform to detect the physical presence of an object (e.g., an interviewee) at a specific location and/or the forces created by an interview across a general area (e.g., is the user shifting weight from one foot to another, pacing, etc.).

In many applications, the local appliance may take the form of a portable device that is not physically linked to any other hardware. In such applications, the local appliance may take a form similar to a tablet device that may be available for the interviewee (or one assisting the interviewee) to handle the device. For other applications, the local appliance may take the form of a device that may be fixed in place and relatively unmovable. In either case, the local appliance may typically operate in conjunction with a power supply that may include a rechargeable battery (which could be integral with the local appliance) or—typically for fixed applications—an external power converter.

FIG. 2 further illustrates different attributes of an exemplary local appliance constructed in accordance with certain teachings of this disclosure. In the referenced figures, the local appliance may take the form of a robust and rugged tablet. As reflected in FIG. 2 the local appliance may take the form of a handheld tablet that can be held by a user and/or positioned on a stand. As further reflected in the referenced figure, the local appliance may optionally take the form of a tablet that is affixed to a support structure such as an articulated arm. In these and other exemplary embodiments, the local appliance may be physically tethered to another structure to both permit constant powering of the device (and thus reduce dependance on any internal batteries) and/or minimize the potential for loss or misplacement of the local appliance.

As still further reflected in FIG. 2, the local appliance may take the form of a tablet that defines a central display area surrounded by horizontal and vertical bezels. In the depicted example, one or more of the sensors discussed above may be positioned within the horizontal and/or vertical bezels. For example, an eye tracker assembly may be positioned generally across the central area of the lower horizontal bezel, a face streaming camera may be positioned within the upper horizontal bezel, left and right speakers may be positioned at the left and right intersections of the vertical bezels and the lower horizontal bezel, and a microphone array may be positioned partially within the top horizontal bezel and partially within the upper part of the right vertical bezel.

In terms of physical construction, the local appliance may be formed in a variety of ways. In accordance with one approach the local appliance may be formed from several different sub-components or systems. First, a base carrier board is provided that provides structure for enabling base connections between all integrated sensors and detectors and any external peripheral sensors or detectors. The base carrier board may also provide basic system level functions such as power and communication options.

Second, the exemplary architecture may include a pluggable system of a module that provides the general processing and storage functionality and that may provide the hardware necessary to enable base communications. Such a system on module (SOM) may take the form of a basic system that provides the main computing functionality, memory/storage functionality and basic communication and I/O functionality required by the local appliance. It may include built-in Wi-Fi and/or Bluetooth hardware and may further include microphone and speaker interfaces. A standard connector—such as a plug-in connector—may be used to couple the SOM to the base carrier board.

Third, the exemplary architecture may include a group of base peripherals and sensors that may interface with the base carrier board and provide various input signals useful to the system. Note that in the depicted example of FIG. LAB, the base carrier board includes unused slots and/or connectors intended to provide expansion capabilities. Such unused slots and/or connectors may enable rapid addition of new sensors in the future.

Active Mobile Systems: Alternate embodiments of the human interface system are envisioned wherein the human interface system may be designed to be movable during use to enable interviews to be conducted at various locations and at unexpected times. For example, one embodiment of such a system could involve the coupling of a local appliance as described above with a transportation and power system such as a walking robot (e.g., an agile mobile robot such as the SPOT robot available from Boston Dynamics) or a movable cart with wheels. Such a system could then be guided to a particular interviewee at any location and an interview could then be conducted at an unexpected time and location. Such a system is believed to be of benefit because experience has shown that “surprise” interviews can often produce the most candid (and therefore the most likely to be truthful) responses.

Note that the human interface system format for an active mobile system need not take the form of a local appliance and that alternate forms may be used. For example, an active mobile human interface system could take the form of a walking bi-pedal humanoid robot that includes the output devices and the sensors necessary to conduct the desired interview.

Home State assessment System: A still further variant of the human interface system is a variant designed to be used in a “home” environment, such as an individual household, a hospital room, or any location where a low-cost state assessment system is desired. In general, such a system could be designed to take advantage of hardware already typically found in a residential location such as a TV monitor with speakers, a camera and a microphone, and WiFi or Ethernet connectivity. For example, a Home State assessment Device that may take the form of a deck-of-card sized device that is capable of receiving input from a microphone and camera via a standard media (e.g., USB) and that is capable of providing audio and visual output to a TV monitor via a standard interface (e.g., HDMI). The home State assessment Device may also include an input for power and could include a WiFi chipset for communication with a local WiFi network (and subsequent communication with a State assessment Server System) and/or an Ethernet connection. In many applications eye tracking information may be useful or necessary. To accommodate such applications, the Home State assessment Device may also include another input port (e.g., a second USB port) for connection to an eye tracker. The eyer tracker may be associated with the resident's TV to create a basic human interface system.

SmartPhone Systems: Given the increasing complexity of smartphones, alternate versions of the human interface system are envisioned where a smartphone serves as a human interface system. In such embodiments, an application may be provided to run on a smartphone that would use the front, or “selfie” camera as a camera and an eye tracker, the phone microphone as a microphone, and the display and speakers of the smartphone as audio/visual output devices.

Virtual/Augmented Reality Systems: Still further embodiments are envisioned wherein the human interface system utilizes available virtual reality or augmented reality systems to interact with a human interviewee. For example, available apparatus like the virtual reality system OCULUS QUEST 2 could provide audio/visual/kinetic outputs to an interviewee and could be combined with sensors to provide the feedback signals required by the state assessment server system for proper analysis. In still further embodiments, augmented reality interface devices—such as AMAZON's smart specs, MICROSOFT's Hololens 2 headset, or LENOVO's ThinkReality A3 glasses—may be used as all or part of a human interface device.

THE COMMUNICATION LINKS: The communication links between the human interface systems and the state assessment server system may take any suitable form, such as wired connections or wireless connections. In certain embodiments involving mobile devices, the communication channels may include wireless communications with some or all the human interface systems (e.g., through high-speed, high bandwidth 5G connections) coupled with downstream wired connections or further wireless connections.

Encryption: As a security measure, all or part of the data used in a state assessment system constructed in accordance with teachings of this disclosure may be encrypted both as it is communicated across any communication link and as processed within the system. Thus, for example, the data received by the human interface system may be encrypted, the data transmitted from the human interface system to the state assessment server system may be encrypted, and all reports and/or analysis generated by the state assessment server system may be encrypted. Note that any such encryption could be distinct from—or integrated with—the anonymous processes discussed previously.

Thus, for example, one could encrypt data that has not be anonymized (such as a non-anonymized video file). While such a file would be encrypted—in the sense that it would not be readily accessed by those not authorized to receive and view such data—it would not be anatomized because anyone able to decrypt the data file could then use it to identify the unique human associated with the file.

In other embodiments one could both anonymize and encrypt data used by the state assessment system either through separate processing steps or through an integrated process where input non-anonymous data is both anonymized and encrypted through a single process step.

As is known to those sufficiently skilled in the art, in addition to using encryption to protect the data from observation, encryption may be used to authenticate the data. That is to say that a device holding a private encryption key may encrypt that data such that it may be verified as being encrypted by that device at a later time.

THE STATE ASSESSMENT SERVER SYSTEM: In one exemplary embodiment the state assessment server system (or systems) will take the form of a server or multiple servers that communicate with the human interface systems to at least: (a) provide most or all of the information necessary to provide stimuli to the interviewee for an interview; (b) receive detected interview data from the human interview systems; (c) process received detected data in light of the provided stimuli to generate further stimuli interactions with an interviewee and/or to assess and analyze the received signals and to provide a report or indication reflecting the underlying state of the human interview; and/or (d) provide an interface into the state assessment server system that may be used to modify the system, adjust the nature of one or more interviews, directly communicate with a human interface system, monitor an interview in real time, or request to generate various reports. Other functionality may be enabled by or within the state assessment server system.

The precise process by which the state assessment server system assesses the underlying state of a human interviewee may vary significantly. For example, in applications where the system is deployed to detect deception at an airport access point, the state assessment server system may include one or more machine learning models—created through the use of significant test data—that correlate certain received signals from the human interface system with deception. For example, in applications where an interviewee is asked whether they are transporting certain contraband, and a question is posed with a visual depiction of the contraband, the vocal inflection of the interviewee along with an assessment of the interviewee's eye gaze pattern (e.g., are the focusing on or avoiding focusing on the displayed contraband in an unusual manner) can provide an indication about whether the interview is being truthful in their response. Various approaches for detecting the state of a human interviewee using received sensor data are discussed and disclosed, for example, in U.S. Patent Application Publication No. 2013/0266925.

The physical implementation of the state assessment server system may take many forms. In one embodiment the state assessment server system may be a computer server (or group of servers) dedicated solely to the distributed state assessment system. In other embodiments the state assessment server system may be implemented virtually in the cloud such that it is not temporally linked to any specific physical hardware. Hybrid approaches are also envisioned.

### Specific Exemplary Embodiment

A distributed state assessment system as described herein may be implemented in such a manner as to provide several advantages not available from localized systems. For example, such advantages include the ability to retain security with respect to certain state assessment processes and to advance and develop such processes using data obtained from a large variety of sources and locations. Such an approach also enables the most efficient use of hardware and software such that various processing steps can be handled at the most appropriate location within the system.

In such an exemplary embodiment it may be desirable to have all or substantially all analysis of the detection signals received during a human interview processed within the state assessment system such that there is no need to store or retain any of the analytical software or models within the various human interface systems. This may be beneficial because wide access to the analytical software or models could allow a malicious actor to learn details of the system that would allow them to increase the chances that they could develop countermeasures to the system. By utilizing a generally centralized state assessment server system, the most sensitive software and systems may be located (physically and/or virtually) in one or more limited locations where appropriate security measures may be maintained.

In the example described above, the various apparatus required to form the human interface systems may be limited to systems that have relatively little “intelligence” in the sense that the human intelligence systems are primarily vehicles to: (a) receive commands from the state assessment server system to provide certain stimuli to the human interviewee and (b) return signals received from the various detectors and sensors associated with the human interface system that are received during an interview. In such an embodiment, the various human interface devices would not store any received data from the state assessment server systems, from the detectors and sensors that comprise the human interface unit, or have any processing capability to self-generate questions, images, sounds or other stimuli to be provided to the interviewee or to perform any state assessment analysis of the signals and detections received by the human interface system during the interview. The human interface system, in such an example, would primarily be a non-intelligent conduit through which the state assessment server system would communicate with the human interviewees. In the example under discussion, all the “intelligence” within the system—specifically the generation of the specific questions and other stimuli to be presented to a human interviewee, the determination of the particular form that a virtual depicted interviewer may take (in terms of appearance, voice, dress, mannerisms, etc.) would all be determined and controlled by the state assessment server system. So too would all the processing and analysis of the detected and sensed signals received during an interview and the reporting of the results of such processing and analysis.

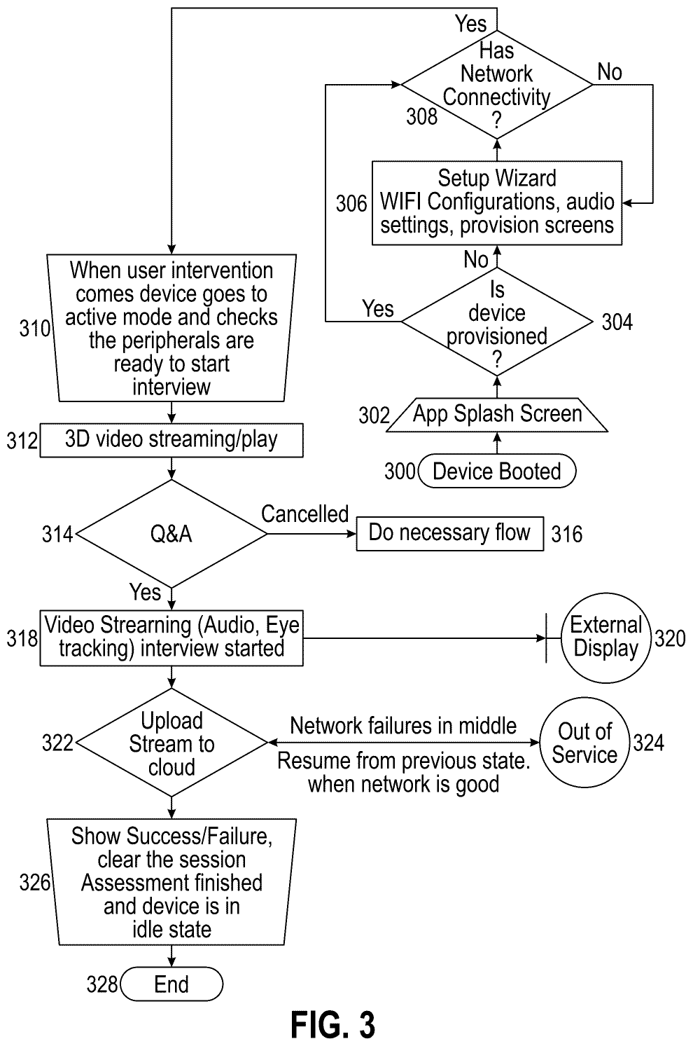

In the exemplary example discussed above, where the various human interface systems take the form of thin clients, the overall operating process for an exemplary human interface system could follow the process illustrated by FIG. 3. FIG. 3 illustrates the high-level general operating process of an exemplary distributed state assessment system with respect to a given human interface unit.

Referring to FIG. 3, the exemplary process begins at a step where the human interface system boots up, typically through a powering on of the apparatus after a state where it is off and non-operable. After the initial boot operation, the human interface system (which in the present example will be discussed in the context of an integrated, generally mobile tablet device) may display a splash screen in which a user or operator of the system may then enable the device through a provisioning step where the specific device is coupled to one or more communication networks, associated with one or more peripheral devices (e.g., a force platform, printer, etc.) The human interface may then seek to make communication contact with the state assessment server system and, once suitable contact and communications have been made, transition to a state where it is ready to begin a human interview.

Once placed in a “ready to start interview” state the human interface system may need to determine when to start the interview.

In one of many simple approaches, the interview session may be initiated by the interviewee interacting with the local appliance by, for example, activating a touch-screen button. In such an exemplary system, the individual to be interviewed may then be instructed to move to a position where they are within the appropriate detectable area of the sensors and detectors (e.g., guided to move to a spot where their face is properly framed within a video camera) and the interview may then be conducted through the provision of stimuli to the interviewee and the receipt and transmission of detector and sensor values. Once the interview is completed, the interview data may be “wiped” form the human interface system, such that no localized record of the interview is maintained, and a report or analysis of the interview may be provided by the state assessment server system to authorized users of the system. The report or analysis may be provided in near-real time via electronic devices, or via delayed communications (e.g., emailed or texted reports reflecting the results of a number of different human interviews.

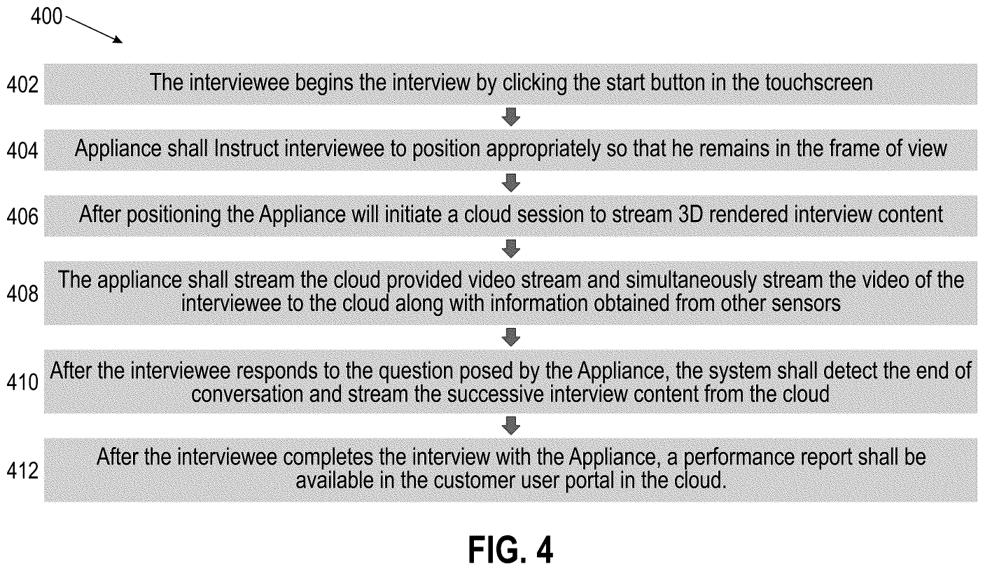

FIG. 4 illustrates an exemplary interview process using a touchscreen start button to start the interview and where the virtual interview agent presented to the interviewee is revered to as AVATAR.

In some alternate embodiments, a human interface system in the form of a local appliance may be adapted to automatically initiate a session upon the occurrence of one or more events. As an example, embodiments are envisioned where the interview is initiated through the use of an “are eyes found” function within the eye tracker. In such embodiments, the receipt of a signal indicating that the eyer tracker for a particular user appliance has detected the presence of eyes may cause the initiation of an interview with respect to that user appliance.

Still further embodiments are envisioned where the occurrence of a multitude of events is required before the initiation of an interview. For example, embodiments are envisioned wherein the simultaneous detection of a weight above a minimum (through the use of a force platform) and the receipt of a positive “eyes found” signal from the eye tracker may be required for the initiation of an interview.

As may be envisioned by those sufficiently skilled in possession of the teachings and disclosures contained herein, the occurrence of a multitude of events may be required before the initiation of an interview.

Calibration: As described generally above, the initial parts of an interview may involve a form of calibration where the human interviewee is asked to physically move in such a manner as to better interact with the human interface system. Other calibration processes are envisioned. For example, to determine the appropriate operation of eye detecting and eye tracking apparatus a human interviewee may be shown certain images on a display (e.g., dots) and asked to look at and track the dots as they move.

While calibration processes as described herein may be used to physically calibrate the system, certain other interactions with the interviewee may be used to “calibrate” the way the data collected during the interview is processed and analyzed by the detection server system. For example, in certain embodiments the state assessment server system may consider the amount of time that passes from the end of a question posed to an interviewee and the start of the interviewee's answering of the question as a data point in assessing the state of the interviewee. While such data may be of significance to an interviewee who is presented questions in (and asked to answer in) their language, such data may not have the same significance for an interviewee questioned in a non-native language. As such, before the substantive portion of an interview is begun, the human interface system may present to the interviewee one or more calibration-type questions (e.g., “Is English your native language?”) to help calibrate or tune the analytical or processing operations performed by the state assessment server system.

Automatic Advancement of the Interview: One issue that a system, such as the described exemplary embodiment, may have to address is when to advance from one question to another. One of many possible approaches would be to have a “next question” button available to the interviewee or to have the interviewee perform some specific detectible gesture (e.g., tap left foot) to move to the next question. However, the requirement for such actions by the interviewee may potentially disrupt the interview process and make it more difficult for the processes running on the state assessment server system to accurately determine the state of the interview. Accordingly, embodiments are envisioned where the distributed state assessment system automatically advances the questions presented to the interview.

Automatic advancement may be accomplished by having the system: detect delays in speech or identify certain words of linguistic patterns indicative that the interviewee has completed their answer to a previous question. In another envisioned embodiment, a new question may be asked by the discern server system before the interviewee has completed answering the current question. This process may be used to disrupt the pace of questions and answers to throw off the interviewee to illicit more honest answers.

Determining Interviewee Consistency: To enhance the viability of the disclosed systems it may be beneficial to confirm, substantially or at several discrete points during an interview, that the interviewee is a single specific individual and that the person acting as the interviewee has not changed during the interview. Such confirmation may detect for example and without limitation, whether a person who has responded to one or more interview questions has been replaced with a different individual at some point during the interview.

Various approaches are envisioned for ensuring interviewee identify consistency. According to certain approaches the apparatus and methods used to ensure interviewee identity consistency may be all located or implemented near the location where the relevant local appliance is located. According to other approaches, the detection apparatuses may be located near the local appliance, but the processing may be done at the central server. In still other approaches, the apparatuses and processes are distributed, with some being located near the local appliance and others apart.

One approach for ensuring identity interview consistence may be to use the camera to periodically or continuously assess the body physique of the interviewee and provide an alarm indication if the detected body physique of the interview changes in an unexpected manner or moves out of the frame.

In embodiments where a force platform is used, as an alternative to, or in addition to the processes described herein, interviewee consistency may be assessed by monitoring the output of the force platform to observe movements that are either unexpected or consistence with a change in the interviewee and/or for an unexpected change of weight during the interview. Upon the detection of such an unexpected event, an alarm or indication reflecting a potential interviewee identity change may be raised.

In addition to confirming the consistent identity of the interviewee, the distributed state assessment system may further implement processes to determine that the interviewee is not inappropriately influenced by others during the interview. For example, the distributed state assessment system—either through a local process run at the human interface system or through a process run at the state assessment server system—may seek to detect: (a) non-interviewee speech of the type that would suggest that a third-party is providing answers to the interviewee; (b) third-party appearance in the frame of the camera; (c) unexpected changes in the detected weight on a force platform (suggesting third-party appearance) or any other indicia of interference.

DISTRIBUTED PROCESSING: One important benefit that may be provided by a distributed processing system constructed in accordance with certain teachings of this disclosure may be that the processing operations necessary to implement the system may be distributed across the various devices comprising the system.

For example, the initial calibration steps, the initial “interview start” detection, and the interviewee consistency processes may all be implemented at a local level through processes operating on the various human interface systems. One benefit of this embodiment may be that it can reduce the extent of information that needs to be transferred from the local appliance to the central server, thus freeing up bandwidth that may be used for the communication of other information or reducing the overall bandwidth requirements. For example, in such embodiments, the local appliance may need only to communicate an alert signal to the central server if its local processing detects a change (or potential change) in the interviewee identity,

In one preferred embodiment, the questions may be presented to the interviewee using a three-dimensional video representation of a human agent. There are various ways in which the video feed associated with the human agent may be generated and presented to the interviewee.

In accordance with one embodiment, the video data for each question (including the video data necessary to present the animated human agent to the interviewee) may be generated in any suitable manner and stored and maintained in a memory system accessible to state assessment server system. In such embodiments, each time a question or instruction is to be presented to an interviewee, the distributed server system will stream the video feed (and potentially the accompanying audio feed) to the appropriate human interface system associated with that interview.

The described embodiment may require the maintenance of many pre-recorded video segments and, in instances where there are a large number of local appliances involved in a large number of individual interviews, the need for the state assessment server system to access and transmit a large number of video segments to the local appliances. This may give rise to storage and bandwidth issues.

Accordingly, alternate embodiments are envisioned where the video (and potentially audio) segments associated with an instruction or question to be presented to an interviewee are rendered in substantially real time by the distributed server system. This embodiment, therefore, reduces the need for the remote storage of various pre-rendered video clips. This embodiment, however, still may have a need for transmission of the rendered video segments to each of the individual human interface systems and, therefore, a need for substantial bandwidth.

Yet a further embodiment is envisioned wherein all of the video rendering is done within the human interface systems, e.g., within local appliances. In this embodiment, the state assessment server system provides each of the human interface systems (e.g., each local appliance) with the data required to render a video (and potentially an audio/visual) presentation of all or various parts of the interview.

For example, in such an embodiment, each human interface system could be programmed—at initial provisioning—with a robust 3D model of a virtual human interviewer. Such a model may take the form, for example, of a human model created using a program such as UNREAL ENGINE's MetaHuman Creator program. After such provisioning, the state assessment server system need only then provide the data files necessary to “animate” the virtual human interviewer to conduct the desired interview. The local human interface systems would then render the received data to generate the image and other associated stimuli provided to the interviewee during the interview.

One advantage of the local rendering approach described herein is that it may significantly reduce the overall bandwidth necessary for the communication channels. In such embodiments, the communication channels need not support significant data transfers between the local human interface systems and the state assessment server system(s) which may therefore enable a given system to support many individual human interface systems (making the overall system very scalable) and/or enable the proper sizing of a system for a given desired workload to reduce cost and promote efficiency. In other words, in certain systems the processing required by the system may be distributed between the local devices and the state assessment server system so that the overall costs of the system may be minimized.

A further advantage of the distributed approach described above is that it may permit readily tailoring of the system to specific applications. For example, in an application where the described system will be used in an international airport—and where individuals of various cultural backgrounds and native languages will be traveling—different human interface systems may be provisioned to provide differently appearing virtual agents for interacting with passengers form different locations. Such different virtual agents could be tailored—in appearance, voice pitch/tone, etc.—to passengers traveling to/from specific countries or regions since, in such an embodiment, the rendering of the virtual agent data file would all be done locally, the state assessment server system may not have to bear the burden of rendering multiple different appearing agents and the communication may not have to bear the data traffic associated with such actions.

A further advantage of the distributed approach described above may be that it allows various authorized users to interact with the distributed state assessment system in various ways. For example, in applications where such a system is used for employment pre-screening and post-employment evaluation, the distributed state assessment system could allow authorized administrators to review various interview data and reports suitable for their roles. For example, a given manager may be able to review select post-employment interview data only for employees under their supervision, while a company-wide security officer may have access to all employee interview data.

A combination of very thin clients with a distributed system may be advantageous in some situations. For example, an entry gate at a sporting event may have multiple queues for people to enter. In this exemplary embodiment, one distributed state assessment system may be placed at the gate for one queue, and several other thin client state assessment systems may service the other queues. Each of the thin client state assessment systems would be linked via a high-speed network to the nearby distributed state assessment system so they could each draw on the resources of the distributed state assessment system. The resources that the distributed state assessment system may offer to the thin client systems may include but would not be limited to: a link to a cloud-based processing system; a codec for coding/decoding video streams; encryption services; memory and processing services.

Another process in which a distributed state assessment system of the type disclosed herein could be used is in connection with medical or mental health evaluation and/or treatment. For example, for individuals who may be subject to state changes resulting from a change in their state (e.g., as a result of a manic incident, a stroke, significant anxiety, failure to take medications, etc.) a home-based human interface system could be used that could report to a licensed professional or an authorized family member both: (a) that the individual using the device has completed a regularly-scheduled interview and (b) whether the results of that interview suggest that the individual is in a state requiring follow-up action or attention. For example, children and others subject to manic states are often prescribed medication to take on a daily basis. Subjecting such interviewees to daily interviews using a home-based human interface system could allow regular and automatic assessment of whether each individual is compliant with their medications and/or whether the medications are operating properly. If the interview suggests that the individual has not taken their medication—or that the interviewee is experiencing a manic state—notice that further action is warranted could be provided.

Note that the above-described example is one where the use of a virtual interviewer could permit the system to be uniquely tailored for use with children. For example, while a child may be hesitant to interact with a stern-appearing virtual interviewer, a virtual interviewer uniquely tailored to that child, such as a cartoonish character in the form of their favorite animal, may result in a situation where the child is both comfortable and eager to participate in an interview as described above.

Note that the described system may also enable the distributed state assessment system of the present invention to be used in a therapeutic manner. For example, if an interview indicates that the interviewee is experiencing sever anxiety, the system could—in addition to alerting the appropriate professional—be designed to respond empathetically (e.g., “You seem upset. Is there something that you want to share?” or “You appear angry. Have you remembered to take your medication this morning?”).

Other and further embodiments utilizing one or more aspects of the inventions described above can be devised without departing from the spirit of Applicant's invention. Further, the various methods and embodiments of the methods of manufacture and assembly of the system, as well as location specifications, can be included in combination with each other to produce variations of the disclosed methods and embodiments. Discussion of singular elements can include plural elements and vice versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components that have multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intend to protect fully all such modifications and improvements that come within the scope or range of equivalent of the following claims.

What is claimed is:

1. A distributed system for conducting an automatic behavioral analysis for the assessment of the mental state of a human interviewee, the system comprising:
at least one human interface system, the at least one human interface system comprising sensors for generating interview data associated with exhibited attributes of the interviewee;
at least one state assessment server system, the at least one state assessment server system comprising a database storing data generated during a plurality of interviews of a given interviewee at different times;
a communication link permitting bi-directional communications between the at least one human interface system and the at least one state assessment server system; and
wherein the at least one state assessment server system is configured to identify at least one pattern within the stored data to predict the onset of a manic/depressive episode for the interviewee.

2. The system of claim 1, wherein the at least one human interface system is configured to present an embodied conversational agent to the interviewee.

3. The system of claim 2, wherein the embodied conversational agent is based on the age of the interviewee.

4. The system of claim 3, wherein the embodied conversational agent takes a form of a non-human agent with which a child may easily interact.

5. The system of claim 1, wherein the interview data comprises data associated with one or more of the following interviewee attributes: kinesics; heart rate, vocalics; and/or oculometries.

6. The system of claim 1, wherein the human interface device is a tablet.
